(19) United States
(12) Patent Application Publication
(10) Pub. No.: US 2022/0002664 A1
(43) Pub. Date: Jan. 6, 2022

(54) LACTOBACILLUS REUTERI LM1071 FROM BREAST MILK HAVING HIGH SAFETY AND INTESTINE ADHESIVE PROPERTY, AND COMPOSITION COMPRISING THE STRAIN OR ITS CULTURE FLUID

(71) Applicant: LACTOMASON CO., LTD., Jinju-si Gyeongsangnam-do (KR)

(72) Inventors: Min Gyu JEON, Jinju-si, Gyeongsangnam-do (KR); Tae Rahk KIM, Suwon-si, Gyeonggi-do (KR); Su Bin JO, Jinju-si, Gyeongsangnam-do (KR); Soo Yeon JEONG, Jinju-si, Gyeongsangnam-do (KR)

(21) Appl. No.: 15/734,909

(22) PCT Filed: Sep. 25, 2020
(86) PCT No.: PCT/KR2020/013085

§ 371 Date: Dec. 3, 2020

(30) Foreign Application Priority Data
Mar. 4, 2020 (KR) 10-2020-0027335

(51) Int. Cl. C12N 1/20

(52) U.S. Cl. CPC C12N 1/205; C12R 2001/225

(57) ABSTRACT

The present disclosure relates to Lactobacillus reuteri LM1071 (KCCM12650P) and a composition containing Lactobacillus reuteri LM1071 for improving the intestinal environment.

Lactobacillus reuteri LM1071 (KCCM12650P) according to an embodiment of the present disclosure does not have hemolysis and biogenic amine production capability and has excellent biosafety and excellent intestine adhesion and thus can improve the intestinal environment and improve the intestinal health. Therefore, the strain can be applied to pharmaceutical compositions, food compositions, health functional food compositions, feed compositions, and the like.

### TECHNICAL FIELD

The present disclosure relates to Lactobacillus reuteri LM1071 (KCCM12650P) and a composition containing Lactobacillus reuteri LM1071 for improving the intestinal environment.

### BACKGROUND

Lactobacillus is in a symbiotic relationship with human within the digestive system, and functions to decompose fiber and conjugated proteins into important nutrients. Live microorganisms that improve the host's microbial environment in the gastrointestinal tract of animals including humans and have beneficial effects on the host's health are collectively referred to as “probiotics”. Representative examples of the probiotics may include Lactobacillus sp. and Lactococcus sp. The probiotics have beneficial effects on human health, such as improvement in immune function, prevention of infection, improvement in mineral absorption, prevention of intestinal disorders by inhibiting intestinal harmful bacteria, alleviation of symptoms of lactose intolerance, prevention of constipation, reduction of blood cholesterol level, prevention of tumor development, prevention of diabetes, and lowering of blood pressure.

In order for strains to be considered as probiotics, they must survive exposure to gastric acid and bile acid, reach the small intestines, and adhere to the intestinal epithelial cells. They must have positive effects such as anti-bacterial and anti-inflammatory properties without exhibiting any toxicity or pathogenicity. The safety and functionality of probiotics have been variously studied so far.

Microbiota refer to the microbial communities in the environment as a microbial flora, and microbiota have been found to be crucial for immunologic and metabolic homeostasis of their host, e.g., human. Microbiota and the host directly interact with each other or indirectly transmit and receive chemical signals to and from each other, and the expression of immune cells, neurotransmitter production and short-chain fatty acids (SCFA) caused by microbiota have a significant effect on the host system. Particularly, probiotics/prebiotics balance the host's unbalanced microbiota so that healthy metabolites of microbiota can improve the host's health.

As results from the study of compositions for improving the intestinal environment, a composition for improving intestinal health comprising a water extract of red yeast rice (Korean Patent No. 10-185030) and a composition for improving intestinal health containing an ethanol extract of Codonopsis lanceolata (Korean Patent No. 10-1826673) are disclosed. However, further research and development of compositions having excellent effect in improving the intestinal environment and treating intestinal diseases is still needed.

Accordingly, the present inventors have tried to develop a composition helpful in improving the intestinal environment and improving the intestinal health and resultantly developed a novel strain that has low hemolysis and biogenic amine production capability and thus has high safety with improved intestine adhesion, and completed the present disclosure.

### DISCLOSURE OF THE INVENTION

### Problems to be Solved by the Invention

The present disclosure relates to Lactobacillus reuteri LM1071 (KCCM12650P) and a composition containing Lactobacillus reuteri LM1071 for improving the intestinal environment.

However, problems to be solved by the present disclosure are not limited to the above-described problems. Although not described herein, other problems to be solved by the present disclosure can be clearly understood by a person with ordinary skill in the art from the following description.

### Means for Solving the Problems

A first aspect of the present disclosure provides Lactobacillus reuteri LM1071 (KCCM12650P).

A second aspect of the present disclosure provides a composition for improving the intestinal environment, containing Lactobacillus reuteri LM1071 (KCCM12650P) or its culture fluid.

### Effects of the Invention

Lactobacillus reuteri LM1071 (KCCM12650P) according to an embodiment of the present disclosure does not have hemolysis and biogenic amine production capability and has excellent biosafety and excellent intestine adhesion and thus can improve the intestinal environment and improve the intestinal health. Therefore, the strain can be applied to pharmaceutical compositions, food compositions, health functional food compositions, feed compositions, and the like.

### BRIEF DESCRIPTION OF THE DRAWINGS

FIG. 1 shows the result of checking the adhesion of LM1071 of the present disclosure to HT-29 cells.

FIG. 2 shows the result of MATS (Microbial Adhesion To Solvents) test of LM1071 according to the present disclosure.

### BEST MODE FOR CARRYING OUT THE INVENTION

Hereafter, examples of the present disclosure will be described in detail with reference to the accompanying drawings so that the present disclosure may be readily implemented by a person with ordinary skill in the art. However, it is to be noted that the present disclosure is not limited to the examples but can be embodied in various other ways. In the drawings, parts irrelevant to the description are omitted for the simplicity of explanation, and like reference numerals denote like parts through the whole document.

Throughout this document, the term “connected to” may be used to designate a connection or coupling of one element to another element and includes both an element being “directly connected to” another element and an element being “electronically connected to” another element via another element.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise. Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through the whole document, a phrase in the form “A and/or B” means “A or B, or A and B”.

Hereinafter, embodiments and examples of the present disclosure will be described in detail. However, the present disclosure may not be limited to the following embodiments and examples.

A first aspect of the present disclosure provides Lactobacillus reuteri LM1071 (KCCM12650P).

In an embodiment of the present disclosure, the strain may have γ-hemolytic activity.

Through the whole document, the term “hemolysis” or “hemolytic activity” is pathogenicity in which red blood cells are ruptured by certain pathogenic bacteria or disease. The degree of hemolysis is separated into α-hemolysis that means partial hemolysis, β-hemolysis that means complete hemolysis and γ-hemolysis that means no hemolytic activity. Pathogenic bacteria or fungi that cause hemocytolysis, i.e., has hemolysis can produce hemolysins that damage the red blood cell's cytoplasmic membrane, causing septicaemia. Therefore, hemolysis must be checked to select a strain to be used as probiotics, and a strain without hemolysis may particularly mean that the strain has excellent biosafety.

In an embodiment of the present disclosure, the strain has γ-hemolytic activity, which means that the strain does not have hemolysis and has in vivo safety and thus can be used in various compositions.

In an embodiment of the present disclosure, the strain does not produce biogenic amines, and specifically, the biogenic amines may include one or more selected from the group consisting of cadaverine, tyramine, histamine and putrescine, but may not be limited thereto.

Through the whole document, the term “biogenic amines” refers to low molecular weight organic nitrogen compounds that are produced when an α-carboxyl group is removed from amino acid and are known as substances that are present in vivo and mediate or regulate physiological functions. Also, the biogenic amines are known to cause toxicological events such as headache, hypertension, pyrexia and heart disease. Examples of major biogenic amines may include cadaverine, tyramine, histamine and putrescine, and these biogenic amines are synthesized from lysine, tyrosine, histidine or ornithine, respectively. Since toxic biogenic amines show various negative effects in vivo, biogenic amine production properties need to be checked to select a strain to be used as probiotics.

In an embodiment of the present disclosure, the strain may have high sensitivity or susceptibility to antibiotics, and specifically, the antibiotics may include one or more selected from the group consisting of ampicillin, erythromycin, gentamicin, tetracycline, streptomycin, vancomycin, chloramphenicol, kanamycin and clindamycin, but may not be limited thereto.

In an embodiment of the present disclosure, the strain having high sensitivity or susceptibility to antibiotics may mean that the minimal inhibitory concentration (MIC) of the strain to antibiotics is lower than a breakpoint set by the European Food Safety Authority (EFSA). The MIC is regarded as the most basic indicator of antibacterial activity and defined as the lowest concentration of a given antibiotic at which the growth of the tested microorganism was suppressed in an sensitivity test in vitro. Also, the EFSA breakpoint is the criteria used in the assessment of bacterial resistance to antibiotics of human or veterinary importance, and the tested microorganism should not grow at a given breakpoint level.

In an embodiment of the present disclosure, the strain of the present disclosure may have an MIC of less than 2 mg/L against ampicillin, an MIC of less than 1 mg/L against erythromycin, an MIC of less than 8 mg/L against gentamicin, an MIC of less than 16 mg/L against tetracycline, an MIC of less than 64 mg/L against streptomycin, an MIC of less than 4 mg/L against chloramphenicol, an MIC of less than 64 mg/L against kanamycin, or an MIC of less than 1 mg/L against clindamycin. Specifically, the strain of the present disclosure may have an MIC of 0.5 mg/L or less against ampicillin, an MIC of 0.25 mg/L or less against erythromycin, an MIC of 2 mg/L against gentamicin, an MIC of 1 mg/L against tetracycline, an MIC of 0.25 mg/L or less against streptomycin, an MIC of 0.5 mg/L or less against chloramphenicol, an MIC of 8 mg/L or less against kanamycin, or an MIC of 1 mg/L or less against clindamycin.

In an embodiment of the present disclosure, the strain has low resistance to antibiotics, and, thus, it is determined that concerns caused by transfer of antibiotic-resistant genes are not likely to occur.

In an embodiment of the present disclosure, the strain may have excellent intestine adhesion.

In an embodiment of the present disclosure, the strain has excellent intestine adhesion and thus can inhibit adhesion of harmful bacteria to the intestinal barrier while creating the environment for the proliferation of beneficial bacteria and enhance the barrier to form a gastrointestinal barrier (GIB), thereby allowing only beneficial nutrients to be absorbed by the intestinal epithelial cells and harmful substances to be filtered out. Also, the strain has excellent intestine adhesion and thus can adhere to the intestinal epithelial cells and synthesize/secrete antimicrobial bacteriocin, lactic acid, organic acid, hydrogen peroxide and the like and thus have excellent antibacterial activity against intestinal harmful bacteria, such as colon bacillus, typhoid, paratyphoid, dysentery bacteria and cholera bacteria, and Helicobacter pylori that causes gastroenteritis and gastric cancer.

In an embodiment of the present disclosure, the strain may have superior intestine adhesion to Lactobacillus rhamnosus GG (LGG).

In an embodiment of the present disclosure, the strain may have hydrophobic properties, and specifically, the surface of the strain may have hydrophobic properties.

In an embodiment of the present disclosure, the strain may have a hydrophobicity of 60% or more in a hexadecane solvent, and specifically, the strain may have a superior hydrophobicity to Lactobacillus rhamnosus GG (LGG).

In an embodiment of the present disclosure, the strain may be excellent in adhering or sticking to the intestinal epithelial cells.

In an embodiment of the present disclosure, the strain may be helpful in improving the intestinal environment or improving the intestinal health, and specifically, the strain may be contained in pharmaceutical compositions, food compositions, cosmetic compositions, health functional food compositions and feed compositions for intestinal environment improvement.

A second aspect of the present disclosure provides a composition for improving the intestinal environment, containing Lactobacillus reuteri LM1071 (KCCM12650P) or its culture fluid. The features described above in respect of the first aspect of the present disclosure may equally apply to the composition according to the second aspect of the present disclosure.

Through the whole document, the term “improvement” refers to all activities enhancing or making better the intestinal environment by administering the composition.

Through the whole document, the term “intestinal environment improvement” refers to advantageously changing the composition of intestinal microbiota and metabolites of the microbiota, improving the intestinal microbiota or improving the intestinal function and intestinal health. The intestinal environment improvement results in an increase in beneficial intestinal bacteria and metabolites of the beneficial bacteria and thus has effects such as vitamin synthesis, improvement of digestion and absorption, prevention of infection, antibacterial activity, immunoregulation and immunopotentiation, and also results in a decrease in harmful bacteria and metabolites of the harmful bacteria and thus has effects such as decrease in intestinal decomposition, decrease in bacterial toxins and decrease in carcinogens. Also, the intestinal environment improvement can prevent or treat intestinal diseases such as diarrhea, constipation and enteritis and also prevent or treat cancers, obesity, diabetes, and brain-related diseases.

In an embodiment of the present disclosure, the intestinal environment improvement may include one or more selected from the group consisting of an increase in microbial diversity of microbiota, an improvement of intestinal microbiota, an enhancement of beneficial intestinal bacteria, a suppression of harmful intestinal bacteria, a regulation of intestinal immunity, an enhancement of antibacterial activity, an improvement and enhancement of intestinal health, a decrease in endotoxin and hydrogen sulfide derived from intestinal microbiota, an increase in metabolites derived from beneficial microbiota, an increase in kind and number of beneficial bacteria and a decrease in kind and number of harmful bacteria, but may not be limited thereto.

In an embodiment of the present disclosure, the composition may be a composition for improving intestinal microbiota, a composition for enhancing the growth of beneficial intestinal bacteria, a composition for suppressing the growth of harmful intestinal bacteria, a composition for enhancing intestinal immunity or a composition for improving intestinal health.

In an embodiment of the present disclosure, the composition may contain Lactobacillus reuteri LM1071, live bodies, heat-killed bodies, culture fluid, fragmented products and/or or extracts thereof.

Through the whole document, the term “heat-killed bacteria” is opposite to the term “live bacteria” and refers to bodies obtained by suppressing the growth of bacteria such as heat-treating live bacteria obtained by fermentation and metabolites thereof. The heat-killed bacteria may contain cytoplasm, cell wall, antibacterial substances such as bacteriocin, polysaccharides, organic acid, and the like. Products using the heat-killed bacteria have higher stability than live bacteria products and are particularly excellent in heat resistance and have high stability to the external environment. Therefore, the products using the heat-killed bacteria are easier to store and have longer shelf life than the existing live bacteria products. Further, since the regulations on the use of antibiotics become stricter, there are a handful of companies that have produced heat-killed bacteria products. Therefore, considering the application as substitutes and the number of the producing companies, the marketability and growth potential is very high.

Through the whole document, the term “culture fluid” refers to a substance obtained by culturing the strain of the present disclosure in a known liquid medium or solid medium and may be interchangeably used with “culture”.

In an embodiment of the present disclosure, the composition may be a food composition or a health functional food composition.

Through the whole document, the term “food” may include meats, sausages, breads, chocolates, candies, snacks, cookies, pizza, ramens, other noodles, gums, dairy products including ice cream, soups, beverages, tea, drinks, alcohol drinks, vitamin complexes, health functional food and health food, and may include all foods in the accepted meaning.

Through the whole document, the term “health functional food” refers to foods prepared and processed using raw materials or ingredients having useful functions in accordance with Korean Health Functional Foods Act, No. 6727, which means that it is ingested for the purpose of obtaining a beneficial effect for health use such as control of nutrients or physiological action for the structure and function of the human body.

The food of the present disclosure can be manufactured by conventional methods used in the art, and can be manufactured by adding conventional raw materials and ingredients used in the art. Further, a formulation of the food is not limited as long as the formulation is accepted as a food. The food composition of the present disclosure may be prepared as a variety of formulations. Since the food is used as raw materials, unlike general drugs, the food composition is free from side effects which may occur when a drug is taken for a long time, and may have excellent portability. Therefore, the food of the present disclosure may be taken as a supplement for enhancing the effects of improving the intestinal environment.

The health food refers to a food having effects of actively maintaining or promoting health conditions, as compared with general foods, and a health supplement food refers to a food for supplementing health. If necessary, the health functional food, health food, and health supplement food may be interchangeably used with each other. Specifically, the health functional food is a food prepared by adding Lactobacillus reuteri LM1071 of the present disclosure to food materials such as beverages, teas, spices, gums, confectionery, etc., or prepared as a capsule, a powder, a suspension, etc. The health functional food means that it has a specific effect on health when consumed, but unlike general drugs, the health functional food is free from side effects that may occur when a drug is taken for a long time since the food is used as raw materials.

Since the food composition of the present disclosure can be routinely ingested, the food composition is expected to show a high efficacy on the prevention or improvement of the intestinal environment and thus can be very usefully applied.

The food composition may further contain a physiologically acceptable carrier. The kind of the carrier is not particularly limited. Any carrier may be used as long as it is commonly used in the art.

Further, the food composition may further contain additional ingredients that are commonly used in food compositions so as to improve smell, taste, visuality, etc. For example, the food composition may contain vitamins A, C, D, E, B1, B2, B6, B12, niacin, biotin, folate, pantothenic acid, etc. Furthermore, the food composition may also contain minerals such as zinc (Zn), iron (Fe), calcium (Ca), chromium (Cr), magnesium (Mg), manganese (Mn), copper (Cu), etc. Moreover, the food composition may also contain amino acids such as lysine, tryptophane, cysteine, valine, etc.

Further, the food composition may also contain food additives, such as preservatives (potassium sorbate, sodium benzoate, salicylic acid, sodium dehydroacetate, etc.), disinfectants (bleaching powder, higher bleaching powder, sodium hypochlorite, etc.), antioxidants (butylhydroxyanisole (BHA), butylhydroxytoluene (BHT), etc.), colorants (tar color, etc.), color-developing agents (sodium nitrite, etc.), bleaching agents (sodium sulfite), seasonings (monosodium glutamate (MSG), etc.), sweeteners (dulcin, cyclemate, saccharin, sodium, etc.), flavors (vanillin, lactones, etc.), swelling agents (alum, potassium D-bitartrate, etc.), fortifiers, emulsifiers, thickeners (adhesive pastes), film-forming agents, gum base agents, antifoaming agents, solvents, improvers, etc. The additives may be selected and used in an appropriate amount depending on the type of food.

Lactobacillus reuteri LM1071 may be added as it is, or may be used in conjunction with other foods or food ingredients according to a conventional method. The mixing amount of active ingredients may be appropriately determined depending on the purpose of use (prophylactic, health or therapeutic treatment). In general, when a food or a beverage is manufactured, the food composition of the present disclosure may be added in an amount of 50 parts by weight or less, specifically 20 parts by weight or less based on the total weight of the food or the beverage. However, when taken for the purpose of health and hygiene, the food composition may be contained in an amount below the range. In addition, since there is no safety problem, the active ingredients may be used in an amount above the range.

The food composition of the present disclosure may be used as, for example, a health beverage composition, and in this case, the health beverage composition may further contain various flavors or natural carbohydrates, as in common beverages. The natural carbohydrates may include monosaccharides such as glucose and fructose; disaccharides such as maltose and sucrose; polysaccharides such as dextrin and cyclodextrin; and sugar alcohols such as xylitol, sorbitol and erythritol. The sweeteners may be natural sweeteners such as thaumatin or a stevia extract; or synthetic sweeteners such as saccharin or aspartame. The natural carbohydrate may be generally used in an amount of from about 0.01 g to about 0.04 g, and specifically, from about 0.02 g to about 0.03 g based on 100 mL of the health beverage composition of the present disclosure.

In addition, the health beverage composition may contain various nutrients, vitamins, minerals, flavors, colorants, pectic acid and salts thereof, alginic acid and salts thereof, organic acid, protective colloidal thickeners, pH regulators, stabilizers, antiseptics, glycerin, alcohols, carbonating agents, etc. Moreover, the health beverage composition may contain fruit flesh used to prepare natural fruit juices, fruit juice beverages, or vegetable beverages. These ingredients may be used individually or in combination. A proportion of the additives is not critical, but is generally selected from 0.01 part by weight to 0.1 part by weight per 100 parts by weight of the health beverage composition of the present disclosure.

The food composition of the present disclosure may contain Lactobacillus reuteri LM1071 of the present disclosure in a variety of % by weight as long as it can exhibit the effect of improving the intestinal environment. Specifically, Lactobacillus reuteri LM1071 of the present disclosure may be contained in an amount of 0.00001% by weight to 100% by weight or 0.01% by weight to 80% by weight based on the total weight of the food composition, but may not be limited thereto.

A third aspect of the present disclosure provides a composition for improving the intestinal health, containing Lactobacillus reuteri LM1071 (KCCM12650P) or its culture fluid. The features described above in respect of the first and second aspects of the present disclosure may equally apply to the composition according to the third aspect of the present disclosure.

In an embodiment of the present disclosure, the composition may contain Lactobacillus reuteri LM1071, live bodies, heat-killed bodies, culture fluid, fragmented products and/or or extracts thereof.

In an embodiment of the present disclosure, the composition may be a food composition, a health functional food composition or a pharmaceutical composition.

In an embodiment of the present disclosure, the pharmaceutical composition may be formulated and used as formulations for oral administration such as powders, granules, tablets, capsules, suspensions, emulsions, syrups or aerosol, external preparations, suppositories or sterile injection solutions by conventional methods, respectively, but may not be limited thereto.

In an embodiment of the present disclosure, the pharmaceutical composition may be formulated with generally used diluents or excipients such as fillers, extenders, binders, wetting agents, disintegrating agents or surfactants, but may not be limited thereto.

In an embodiment of the present disclosure, solid formulations for oral administration may include tablets, pills, powders, granules or capsules, and these solid formulations may be prepared by mixing heat-killed bodies of Lactobacillus reuteri with at least one of excipients such as starch, calcium carbonate, sucrose, lactose or gelatin. Except for the simple excipients, lubricants such as magnesium stearate or talc may be used, but the present disclosure may not be limited thereto.

In an embodiment of the present disclosure, liquid formulations for oral administration may include suspensions, solutions for internal use, emulsions and syrups, and may contain various excipients such as wetting agents, sweeteners, aromatics and preservatives in addition to generally used simple diluents such as water and liquid paraffin, but may not be limited thereto.

In an embodiment of the present disclosure, formulations for parenteral administration may include sterilized aqueous solutions, water-insoluble excipients, suspensions, emulsions, lyophilized preparations and suppositories, but may not be limited thereto. For example, the water insoluble excipients or suspensions may contain propylene glycol, polyethylene glycol, vegetable oil such as olive oil, injectable ester such as ethylolate, and the like, but may not be limited thereto. For example, the suppositories may contain witepsol, macrogol, tween 61, cacao butter, laurin butter, glycerol, gelatin, and the like, but may not be limited thereto.

The pharmaceutical composition according to an embodiment of the present disclosure may be a drug composition or a quasi-drug composition.

Through the whole document, the term “quasi-drug” refers to an article having a milder action than drugs, among articles being used for the purpose of diagnosis, treatment, improvement, alleviation, handling or prevention of human or animal diseases. For example, according to Pharmaceutical Affairs Law, the quasi-drugs are those, excluding articles used as drugs, including articles used for the purpose of treating or preventing human or animal diseases and articles which have a mild action on or have no direct influence on the human body.

The quasi-drug composition of the present disclosure may be manufactured in a formulation selected from the group consisting of body cleanser, sanitizer, detergent, kitchen cleanser, detergent for cleaning, toothpaste, mouthwash, wet wipe, cleanser, soap, hand soap, hair cleanser, hair softener, humidifying filler, mask, ointment or filter filler, but may not be limited thereto.

In an embodiment of the present disclosure, the pharmaceutical composition may be administered in a pharmaceutically effective amount. Through the whole document, the term “pharmaceutically effective amount” refers to an amount sufficient to treat or prevent diseases at a reasonable benefit/risk ratio applicable to any medical treatment or prevention. An effective dosage level may be determined depending on factors including severity of the disease, drug activity, a patient's age, body weight, health conditions, gender, sensitivity to the drug, administration time, administration route, and excretion rate of the composition of the present disclosure, duration of treatment, drugs blended with or co-administered with the composition of the present disclosure, and other factors known in the medical field. The pharmaceutical composition of the present disclosure may be administered individually or in combination with a known ingredient for treating intestinal diseases. It is important to administer an amount to obtain a maximum effect in a minimum amount without side effects by considering all of the above-described factors.

In an embodiment of the present disclosure, an administration dose of the pharmaceutical composition may be determined by a person with ordinary skill in the art in view of purpose of use, severity of the disease, a patient's age, body weight, gender, medical history or the kind of a material used as an active ingredient. For example, the pharmaceutical composition of the present disclosure may be administered at a dose of from about 0.1 ng/kg to about 1,000 mg/kg, and preferably, from about 1 ng/kg to about 100 mg/kg per adult, and the administration frequency of the composition of the present disclosure is not particularly limited, but the composition may be administered once a day or several times a day in divided doses. The administration dose or the administration frequency does not limit the scope of the present disclosure in any aspect.

Hereinafter, the present disclosure will be explained in more detail with reference to Examples. However, the following Examples are illustrative only for better understanding of the present disclosure but do not limit the present disclosure.

### EXAMPLES

### Example 1: Checking of Hemolysis of Lactobacillus reuteri LM1071

To check hemolysis of a novel strain, Lactobacillus reuteri LM1071 (Depository Institution: Korean Culture Center of Microorganisms, Accession Number: KCCM12650P and Date of Deposit: Dec. 31, 2019; hereinafter, referred to as “LM1071”), of the present disclosure, a test was conducted as described below.

Specifically, LM1071 of the present disclosure was incubated in an MRS medium at 37° C. for 18 hours and then, the incubated strain was treated on an agar plate with 5% sheep blood and incubated at 37° C. for 24 hours. After incubation, β-hemolysis was checked based on the degree of formation of a transparent zone around a colony and E. coli. ATCC 25922 was used as a positive control group.

According to the test result, it can be seen that LM1071 of the present disclosure shows γ-hemolysis that means no hemolytic activity and the positive control group E. coli. ATCC 25922 shows β-hemolysis (Table 1).

TABLE 1
Determination of hemolysis activity

[symbol]

[symbol]

| Strain [symbol] | Hemolysis activity [symbol] | [symbol] |
|---|---|---|
| LM1071 [symbol] | Gamma [symbol] | [symbol] |
| E. coli ATCC 25922 [symbol] | Beta [symbol] | [symbol] |

Based on the above-described result, it can be seen that that LM1071 of the present disclosure does not have hemolysis and thus can be used as probiotics and can be applied to various compositions.

### Example 2: Checking of Biogenic Amine Production Capability of Lactobacillus reuteri LM1071

To check biogenic amine production capability of a novel strain, Lactobacillus reuteri LM1071, of the present disclosure, a test was conducted as described below.

Specifically, LM1071 of the present disclosure was incubated in an MRS medium at 37° C. for 24 hours and then, the incubated strain was incubated on specific agar plates with lysine, tyrosine, histidine and ornithine which are precursors of major biogenic amines [cadaverine, tyramine, histamine and putrescine, respectively] at 37° C. for 24 hours to check whether the biogenic amines were produced. Also, E. coli. ATCC 25922 was used as a positive control group.

According to the test result, it can be seen that the positive control group E. coli. ATCC 25922 produces all the major biogenic amines, i.e., cadaverine, tyramine, histamine and putrescine, whereas LM1071 of the present disclosure does not produce any of the biogenic amines (Table 2).

TABLE 2
Determination of biogenic amines production

[symbol]

[symbol]

| Stain [symbol] | Histamine [symbol] | Cadaverine [symbol] | Tyramine [symbol] | Putrescine [symbol] | [symbol] |
|---|---|---|---|---|---|
| LM1071 [symbol] | − [symbol] | − [symbol] | − [symbol] | − [symbol] | [symbol] |
| E. coli | + [symbol] | + [symbol] | + [symbol] | + [symbol] | [symbol] |
| ATCC | | | | | |
| 25922 [symbol] | | | | | |

+: Positive;
−: Negative

[symbol]

Based on the above-described result, it can be seen that that LM1071 of the present disclosure does not have biogenic amine production capability and thus can be used as probiotics and can be applied to various compositions.

### Example 3: Checking of Antibiotic Sensitivity of Lactobacillus reuteri LM1071

To check antibiotic sensitivity of a novel strain, Lactobacillus reuteri LM1071, of the present disclosure, a test was conducted as described below.

Specifically, LM1071 of the present disclosure was incubated in a mixture of IST broth (90%) and MRS broth (10%) at 37° C. for 48 hours according to the guidelines of the Clinical and Laboratory Standards Institute and then, the minimal inhibitory concentration (MIC) of antibiotics was checked. To check the MIC, a single colony was picked and resuspended in PBS to have an optical density (OD) of 0.01 and inoculated into a plate at 37° C. for 24 hours by using a multipin-inoculator. The MIC point was determined as the lowest concentration of antibiotics at which visible growth is suppressed, as set by the European Food Safety Authority (EFSA).

Since antibiotics have been used to treat diseases caused by pathogenic microbial infection, the development in antibiotic resistance of pathogenic bacteria becomes a great threat. In this regard, according to some studies, it is known that symbiotic bacteria can store antibiotic resistance genes such as pathogenic bacteria and serve as a medium for transferring the antibiotic resistant genes to the pathogenic bacteria and thus play a big part in the development in antibiotic resistance. In recent years, this phenomenon can be observed from lactic acid bacteria. Therefore, it is important to assess the antibiotic sensitivity of lactic acid bacteria used as probiotics.

According to the test result, it can be seen that LM1071 of the present disclosure has a lower MIC to antibiotics, such as ampicillin, erythromycin, gentamicin, tetracycline, streptomycin, vancomycin, chloramphenicol, kanamycin and clindamycin than the concentration suggested by EFSA (Table 3), and, thus, the strain has lower resistance to antibiotics compared with other general strains.

TABLE 3
Determination of minimum inhibitory concentration (MIC)

[symbol]

[symbol]

Antibiotic resistance test

[symbol]

| | Minimum inhibitory concentration (mg/L) of antibiotics [symbol] | | | | | | | | | [symbol] |
|---|---|---|---|---|---|---|---|---|---|---|
| Strain [symbol] | Amp [symbol] | Ery [symbol] | Gen [symbol] | Tet [symbol] | Str [symbol] | Van [symbol] | Chl [symbol] | Kan [symbol] | Cli [symbol] | [symbol] |
| LM1071 [symbol] | ≤0.5 [symbol] | ≤0.25 [symbol] | 2 [symbol] | 1 [symbol] | ≤0.25 [symbol] | — [symbol] | ≤0.5 [symbol] | ≤8 [symbol] | ≤1 [symbol] | [symbol] |
| EFSA breakpoint [symbol] | 2 [symbol] | 1 [symbol] | 8 [symbol] | 16 [symbol] | 64 [symbol] | n.r [symbol] | 4 [symbol] | 64 [symbol] | 1 [symbol] | [symbol] |

Amp: Ampicilin;
Ery: Erythromycin;
Gen: Gentamicin;
Tet: Tetracycline;
Str: Streptomycin;
Van: Vancomycin;
Chl: Chloramphenicol;
Kan: Kanamycin;
Cli: Clindamycin;
n.r: not required

[symbol]

Based on the above-described result, it can be seen that that LM1071 of the present disclosure does not have sensitivity to antibiotics and thus can be used as probiotics and can be applied to various compositions.

### Example 4: Checking of Intestine Adhesion Activity of Lactobacillus reuteri LM1071

To check the intestine adhesion activity of a novel strain, Lactobacillus reuteri LM1071, of the present disclosure, a test was conducted as described below.

First, to check adhesion to HT-29 cells, a human colon adenocarcinoma cell line, the HT-29 cells were incubated in an RPMI1640 medium with 10% fetal bovine serum, 100 Wml of penicillin and 100 meml of streptomycin at 37° C. with 5% CO₂, and 1×10⁵ cells were seeded per well in a 24-well plate to prepare an HT-29 single layer. Thereafter, LM1071 of the present disclosure was diluted with peptone water so as to have an OD of 0.1 and then, the HT-29 cells formed into a single layer were treated with 1 ml of LM1071 of the present disclosure and incubated at 37° C. with 5% CO₂ for 2 hours. After incubation, washing with PBS was performed to remove the bacteria which had not adhered to the HT-29 cells, followed by treatment with 0.25% trypsin-EDTA. To count the number of cells adhering to the HT-29 cells, a pour plate method including diluting a sample treated with trypsin and containing the strain by stages, spraying 1 ml of the diluted sample on a plate, and pouring a cooled MRS agar medium was used. The adhesion activity was expressed as a relative adhesion rate (CFU of adhering strain/CFU of added strain×100). Also, Lactobacillus rhamnosus GG (ATCC53103; hereinafter, referred to as “LGG”) were used as a positive control group.

Meanwhile, approximately 10¹⁴ bacteria live in the human gastrointestinal (GI) tract, and more than 1,000 kinds of bacteria constantly interact with each other. Therefore, the adhesion of bacteria to the slime layer of the GI tract is known as one of important prerequisites for affecting human health.

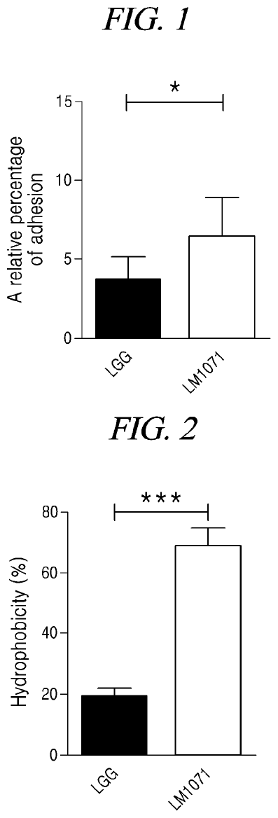

According to the test result, it can be seen that adhesion to the HT-29 cells was remarkably higher in LM1071 of the present disclosure than in LGG as a control group in vitro (FIG. 1).

Then, to measure microbial adhesion to solvents (MATS), LM1071 of the present disclosure was incubated in an MRS medium for 24 hours and then centrifuged at 12,000 rpm for 2 minutes to obtain cell pellets, followed by washing with PBS and dilution to have an OD of 1.0±0.05 (about 1.0×10⁸ CFU/ml) at 600 nm (A₀). The cell suspension was mixed with the same amount of a solvent and then settled at room temperature for 30 minutes, and the absorbance of an aqueous phase liquid layer was measured at 600 nm (A₁). The percentage of adhesion of the strain to the solvent was calculated as (1−A₁/A₀)×100.

Meanwhile, the suppression of pathogenic bacteria is caused by physicochemical properties of the adhesive cell walls of bacteria, and the adhesive properties are affected by interactions such as van der Waals interactions, Lewis acid-base interactions, hydrophobic interactions and electrostatic interactions. Therefore, hydrophobicity occurring at a first contact between bacterial cells and slime or epithelial cells is known as being most important. Further, the intestine adhesion of microbial cells is greatly affected by the structure and composition of the cell surface, and particularly, the hydrophobicity of the cell surface is known as an important factor.

According to the test result, it can be seen that LM1071 of the present disclosure has a superior hydrophobicity in a hexadecane solvent to LGG as a control group (FIG. 2) and the both strains have no difference in hydrophobicity in a xylene solvent.

Based on the above-described result, it can be seen that that LM1071 of the present disclosure has excellent intestine adhesion and thus can be used as probiotics and can be applied to various compositions.

The above description of the present disclosure is provided for the purpose of illustration, and it would be understood by a person with ordinary skill in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

### ACCESSION NUMBER

Depository Institution: Korean Culture Center of Microorganisms (Overseas)

Accession Number: KCCM12650P

Date of Deposit: Dec. 31, 2019

We claim:

1. Lactobacillus reuteri LM1071 (KCCM12650P) having intestine adhesion and sensitivity to antibiotics including one or more selected from the group consisting of ampicillin, erythromycin, gentamicin, tetracycline, streptomycin, vancomycin, chloramphenicol, kanamycin and clindamycin.

2. The Lactobacillus reuteri LM1071 of claim 1,
wherein the strain has γ-hemolytic activity.

3. The Lactobacillus reuteri LM1071 of claim 1,
wherein the strain does not produce biogenic amines.

4. The Lactobacillus reuteri LM1071 of claim 3,
wherein the biogenic amines include one or more selected from the group consisting of cadaverine, tyramine, histamine and putrescine.
